(19) United States
(12) Patent Application Publication
(10) Pub. No.: US 2005/0123621 A1
(43) Pub. Date: Jun. 9, 2005

(54) Silver coatings and methods of manufacture

(75) Inventors: Scott A. Burton, Woodbury, MN (US); Mark J. Hendrickson, Minneapolis, MN (US); Patrick D. Hyde, Burnsville, MN (US); Prabhakara S. Rao, Maplewood, MN (US); Caroline M. Ylitalo, Stillwater, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/728,446

(22) Filed: Dec. 5, 2003

(51) Int. Cl. A61K 33/38; A61L 15/00

(52) U.S. Cl. 424/618; 424/445; 442/123

(57) ABSTRACT

A silver composition containing sparingly soluble silver compounds and a method of coating the composition on a substrate is disclosed.

### BACKGROUND

While wounds heal more effectively in moist environments, bacterial infection poses increased risk. Use of antibiotics to treat bacterial infections can build bacterial resistance. Silver compounds are known to impart antimicrobial effects to a surface with minimal risk of developing bacterial resistance. Silver is delivered to the surface by sustained release of silver ions from the surface when in contact with moist environments, such as a wound bed.

Silver compositions, such as silver nitrate and silver sulfadiazine, are effective antimicrobials used in a variety of applications. However, they are typically not light stable, leave a stain on skin with which they come into contact, and in the case of silver nitrate, can be quickly depleted in an aqueous environment. Wound dressings containing silver antimicrobials include textiles coated with silver compositions, such as those described in U.S. Pat. No. 6,436,420; hydrocolloids prepared with silver-amine complexes, such as those described in U.S. Pat. No. 6,468,521; silver chloride in a wound dressing matrix described in EP 272149; and silver alginate wound dressings described in U.S. 2003/0021832.

Certain silver compounds, such as silver oxides and select silver salts, are both stable and antimicrobial but demonstrate low solubility in aqueous media. Attempts to coat substrates with such compounds have had limited success, leaving limited quantities of the antimicrobial silver compound on the substrate.

### SUMMARY

The present invention is directed to a method of coating silver compounds on a medical article, such as a gauze, a nonwoven, a foam, and a hydrocolloid. The coated silver compositions are preferably stable. By this it is meant that the compositions are stable to at least one of the following types of radiation: visible light, ultraviolet light, electron beam, and gamma ray sterilization.

In one aspect, the present invention provides a method of coating silver compounds on a substrate, comprising combining a sparingly soluble silver-containing compound with an ammonium-containing compound to form a solution, coating the solution on a substrate, and drying the coated substrate. The solution can be formed and/or coated at temperatures less than 40° C. An oxidizing agent can also be added to the solution or the coated substrate.

In another aspect, a method of coating silver compounds on a substrate, comprising combining silver oxide with ammonium carbonate to form a solution, coating the solution on a substrate, and drying the coated substrate. The silver oxide is essentially the only compound that remains on the substrate after drying the substrate, with essentially all of the ammonium-containing compound removed after drying the substrate. An oxidizing agent can also be added to the solution or the coated substrate.

In another aspect, the silver compound can be coated on a substrate such as a nonwoven gauze, a woven gauze, a polyester fiber, a foam, a film and a hydrocolloid. In another aspect, an article is provided that is impregnated with a sparingly soluble silver-containing compound and essentially free of either the ammonium compound or residual components of the ammonium compound and the silver-containing compound.

In another aspect, a method of coating silver compounds on a substrate is provided, comprising combining silver oxide with an ammonium-containing compound to form a solution, adding an oxidizing agent in an effective amount to increase the valence state of the silver oxide, coating the solution on a substrate, and drying the coated substrate.

As used herein, “a,” “an,” “the,” “at least one,” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments.

### DETAILED DESCRIPTION OF ILLUSTRATIVE EMBODIMENTS OF THE INVENTION

The present invention provides a method for coating sparingly soluble silver compounds, such as silver oxides and silver salts, by dissolving silver compounds and ammonium salts in an aqueous solution, coating the solution on a substrate, and drying the coated substrate. The ammonium salts complex with the sparingly soluble silver compounds to allow dissolution in water. Sparingly soluble as used herein can generally be defined as a silver compound concentration in solution of at least 1 μg/gram in water but less than 0.1 g per liter of water.

The process can be accomplished as a continuous process, can be done in a single step or with a single coating solution. The process to apply the coating does not require elevated temperatures, and can be applied at temperatures less than 40° C., and preferably ambient or room temperature, e.g., 23° C. The coating solution can be maintained below a pH of 13, and preferably less than 10, to minimize adverse effects to the substrate.

Sparingly soluble silver compounds provide sustained release of silver ions over time based in part on their limited solubility and inherent dissociation equilibrium constants. Silver compounds useful in the present invention include silver oxide, silver sulfate, silver acetate, silver chloride, silver lactate, silver phosphate, silver stearate, silver thiocyanate and silver carbonate. In a preferred embodiment, the silver compound is silver oxide.

The sparingly soluble silver compounds are dissolved in solution by complexing the silver compound with an ammonium salt. Suitable ammonium salts include ammonium pentaborate, ammonium acetate, ammonium carbonate, ammonium peroxyborate, ammonium tertraborate, triammonium citrate, ammonium carbamate, ammonium bicarbonate, ammonium malate, ammonium nitrate, ammonium nitrite, ammonium succinate, ammonium sulfate, ammonium tartarate, and mixtures thereof. Depending on the silver compound chosen, the silver compound may dissolve easily at room temperature, or may require mechanical action such as stirring over time to aid dissolution when heat is not applied.

The resultant solution containing the silver compound complexed with the ammonium salt can be coated on a substrate, typically an absorbent substrate. The coated substrate is dried to drive off the ammonia and other residual components, such as water and carbon dioxide, for example. Drying can be accomplished at room temperature or by heating the coated substrate. Heat will speed the drying process. In a preferred embodiment, the coated substrate is dried at temperatures below 200° C., and more preferably below 160° C., to minimize decomposition of the silver compounds.

Once dried, the substrate remains coated with the silver compound. The coated substrates are essentially free of silver metal, i.e., Ag(0). In some embodiments, the choice of starting materials results in a coating that leaves no residue with essentially only the silver compound remaining on the substrate, and all other components of the silver solution removed from the substrate upon drying. Preferably, the silver solution is formed from the combination of silver oxide and ammonium carbonate. After coating, ammonia and carbon dioxide are driven off, leaving only the silver oxide remaining on the substrate.

In some embodiments, a higher valence silver oxide, i.e., where the oxidation state of silver is Ag (II), or Ag(III), can be used. The valence state of the silver coated on the substrate can be determined by use of the starting silver oxide material, i.e., AgO, Ag₂O, Ag₂O₃, Ag₂O₄. Alternatively, the valence state of the silver oxide can be increased by the addition of an oxidizing agent to the complexed silver oxide/ammonium salt solution or to the substrate after coating the solution. Suitable oxidizing agents include hydrogen peroxide and alkali metal persulfates such as sodium persulfate, as discussed in U.S. Pat. No. 6,436,420 to Antelman. Other suitable oxidizing agents include permanganates, hypochlorites, perchlorates, and nitric acid.

When applied, the silver solution penetrates and impregnates the interior of the substrate. For example, when gauze is used, the silver solution impregnates between the fibers of the gauze. Similarly, when foam is used as the substrate, the silver solution impregnates the foam cells by both capillary action and absorption into the foam.

The concentration of silver compound on the substrate is a function of the silver compound in solution and the total amount of solution applied onto a unit area of the substrate. The silver compound concentration on the substrate is typically less than 10 mg/cm². In a preferred embodiment, the silver compound concentration on the substrate ranges from 0.1 mg/cm² to 2 mg/cm².

The silver compositions, once coated, are preferably stable. By this it is meant that the compositions are stable to at least one of the following types of radiation: visible light, ultraviolet light, electron beam, and gamma ray sterilization. In certain embodiments, the coated compositions are stable to visible light, such that the coated compositions do not darken upon exposure to visible light. Such compositions are useful in medical articles, particularly wound dressings and wound packing materials, although a wide variety of other products can be coated with the silver compositions. Wound dressings containing hydrocolloids can be used in their hydrated or swollen forms if desired.

Articles can be prepared using the silver solution described herein according to a variety of coating methods. When a porous substrate is coated, the process used typically allows the yarns, filaments, or film such as perforated or microporous film, to be coated, while leaving most of the apertures unobstructed by the composition. Depending on the structure of the support used, the amount of solution employed will vary over a wide range.

According to a variant of this process, a substrate can be passed through a bath of the silver composition. The substrate covered with the silver composition is then dried, for example in an oven at a temperature sufficient to evaporate constituents of the solution. The temperature is preferably at least 100° C.

The silver solution can also be coated onto a carrier web or a backing (described below) using a known coating technique such as gravure coating, curtain coating, die coating, knife coating, roll coating, or spray coating. A preferred coating method is gravure coating.

If desired, compositions of the present invention can be sterilized. Methods of sterilization include treatment with electron beam or gamma radiation.

Medical Articles

The silver compositions of the present invention can be used in a wide variety of products, although they are preferably used in medical articles. Such medical articles can be in the form of a wound dressing, wound packing material, or other material that is applied directly to or contacts a wound. Other potential products include clothing, bedding, masks, dust cloths, shoe inserts, diapers, and hospital materials such as blankets, surgical drapes and gowns.

The silver compositions can be coated on various backings (i.e., a support substrate). The backing or support substrate can be porous or nonporous. The composition of the present invention can be coated on the support substrate or impregnated into it, for example.

Suitable materials are preferably flexible, and may be fabric, non-woven or woven polymeric webs, polymer films, hydrocolloids, foam, metallic foils, paper, and/or combinations thereof. More specifically, cotton gauze is useful with the silver compositions of the present invention. For certain embodiments it is desirable to use a permeable (e.g., with respect to moisture vapor), open apertured substrate (i.e., a scrim). For certain embodiments it is desirable to use an open- or closed-cell foam, such as that disclosed in U.S. Pat. No. 6,548,727. For certain embodiments, the substrate may be a hydrocolloid, such as a hydrophilic polymer, or hydrophobic polymer matrix containing hydrophilic particles, as described in applicants' copending applications, Ser. No. ______, Attorney Docket No. 57260US003 and Ser. No. ______, Attorney Docket No. 59405US002, both of which are incorporated herein by reference.

The substrates (i.e., backings) are preferably porous to allow the passage of wound fluids, moisture vapor, and air. In certain embodiments, the substrates are substantially impervious to liquid, especially wound exudate. In certain embodiments, the substrates are capable of absorbing liquid, especially wound exudate. In certain embodiments, the substrate is an apertured liquid permeable substrate.

Suitable porous substrates include knits, wovens (e.g., cheese cloth and gauze), nonwovens (including spun-bonded nonwovens, and BMF (blown micro fibers), extruded porous sheets, and perforated sheets. The apertures (i.e., openings) in the porous substrates are of sufficient size and sufficient number to facilitate high breathability. For certain embodiments, the porous substrates have at least 1 aperture per square centimeter. For certain embodiments, the porous substrates have no greater than 225 apertures per square centimeter. For certain embodiments, the apertures have an average opening size (i.e., the largest dimension of the opening) of at least 0.1 millimeter (mm). For certain embodiments, the apertures have an average opening size (i.e., the largest dimension of the opening) of no greater than 0.5 cm.

For certain embodiments, the porous substrates have a basis weight of at least 5 grams/meter 2. For certain embodiments, the porous substrates have a basis weight of no greater than 200 grams/meter².

The porous substrates (i.e., backings) are preferably flexible yet resistant to tearing. For certain embodiments, the thickness of the porous substrates is at least 0.0125 mm. For certain embodiments, the thickness of the porous substrates is no greater than 3 mm.

Materials of the backing or support substrate include a wide variety of materials including paper, natural or synthetic fibers, threads and yarns made from materials such as cotton, rayon, wool, hemp, jute, nylon, polyesters, polyacetates, polyacrylics, alginates, ethylene-propylene-diene rubbers, natural rubber, polyesters, polyisobutylenes, polyolefins (e.g., polypropylene polyethylene, ethylene propylene copolymers, and ethylene butylene copolymers), polyurethanes (including polyurethane foams), vinyls including polyvinylchloride and ethylene-vinyl acetate, polyamides, polystyrenes, fiberglass, ceramic fibers, and/or combinations thereof.

The backing can also be provided with stretch-release properties. Stretch-release refers to the property of an adhesive article characterized in that, when the article is pulled from a surface, the article detaches from the surface without leaving significant visible residue. For example, a film backing can be formed from a highly extensible and highly elastic composition that includes elastomeric and thermoplastic A-B-A block copolymers, having a low rubber modulus, a lengthwise elongation to break of at least 200%, and a 50% rubber modulus of not above 2,000 pounds/square inch (13.8 megapascals (MPa)). Such backings are described in U.S. Pat. No. 4,024,312 (Korpman). Alternatively, the backing can be highly extensible and substantially non-recoverable such as those described in U.S. Pat. No. 5,516,581 (Kreckel et al,).

In certain embodiments, the coated substrates of the present invention are nonadherent, although it should be understood that an adhesive (e.g., a pressure sensitive adhesive) could be added to an article coated with the solution. As used herein, the silver compositions of the present invention when coated on a substrate do not adhere significantly to wound tissue such that they do not cause pain and/or destruction of the wound tissue upon removal and display a 180° peel strength of less than 1 N/cm from steel, as described in applicants' copending application, Ser. No. ______, Attorney Docket No. 59098US002, incorporated by reference herein.

In certain embodiments, substrates coated with the silver composition can be covered on one or both sides by a permeable nonadherent outside layer to reduce adhesion and attachment to the wound. The nonadherent layer can be attached to the substrate, such as by coating or laminating. Alternatively, the coated substrate can be enclosed within a nonadherent layer, such as sleeve. The nonadherent layer can be made from nonadherent woven or nonwoven fabrics such as nylon or perflourinated-material coatings on cotton gauze. The nonadherent layer prevents attachment of materials from the enclosed silver coated substrate. At the same time, the nonadherent layer does not adversely affect the sustained release of silver from the coated substrate.

In another embodiment, the backing or support substrate can be composed of nonadherent material. For example, a nonadherent hydrophilic polymer can be used as the backing or support material, or coated on a permeable porous substrate, as described in applicants' copending applications, Ser. No. ______, Attorney Docket No. 57260US003; Ser. No. ______, Attorney Docket No. 59098; and Ser. No. ______, Attorney Docket No. 59405US002.

If desired, the coated substrate can be covered with two protective films (for example, thin polyester films). These films optionally may include a nonstick treatment and can function to facilitate extraction from a package and in handling the article. If desired, the coated substrate can be cut into individual compresses, of sizes suitable for the use, packaged in sealed sachets, and sterilized.

Pressure sensitive adhesives used in medical articles can be used in articles of the present invention. That is, a pressure sensitive adhesive material could be applied to the article of this invention, for example, around the periphery, to adhere the article to the skin.

### EXAMPLES

Objects and advantages of this invention are further illustrated by the following examples, but the particular materials and amounts thereof recited in these examples, as well as other conditions and details, should not be construed to unduly limit this invention. All percentages are in weight percent unless specified otherwise.

Materials

Silver (I) Oxide (Ag₂O), Formula Weight (FW) is 231.7, available from Alfa Aesar, Ward Hill, Massachussetts.

Silver (II) Oxide (AgO), Formula Weight (FW) is 123.9, available from Alfa Aesar, Ward Hill, Massachussetts.

Silver sulfate, Formula Weight (FW) is 311.8, available from Alfa Aesar, Ward Hill, Massachussetts.

Trypticase (Tryptic) Soy Broth (TSB) medium available from Becton Dickinson & Company, Bedford, Mass.

Polyester Knitted Fabric, a 24 mesh polyester knit (1.8 oz/sq yard) purchased from Lamports Filter Media, Inc, Cleveland, Ohio.

Ammonium carbonate, available from Mallinkrodt Baker, Inc., Phillipsburg, N.J.

Ammonium pentaborate, available from Mallinkrodt Baker, Inc., Phillipsburg, N.J.

Cotton nonwoven, 80 g/m², available from Cotton Incorporated, Cary, N.C.

Woven cotton, available from American Fiber and Finishing, Albermarle, N.C.

KRATON Dl 124K—radial 4-arm star polystyrene-polyisoprene (SI)₄ thermoplastic elastomeric copolymer having 30 wt-% polystyrene, available from KRATON Polymers, Houston, Tex.

SALCARE SC95— polymerized methylchloride quaternary ammonium salt of dimethylaminoethylmethacrylate (DMAEMA) dispersed in mineral oil and proprietary non-ionic surfactant, available from Ciba Specialty Chemicals, High Point, N.C.

SALCARE SC91— polymerized sodium acrylate dispersed in mineral oil and proprietary non-ionic surfactant, available from Ciba Specialty Chemicals, High Point, N.C.

KAYDOL—mineral oil available from Crompton Corporation, formerly Witco Corporation.

IRGANOX 1010—Phenolic antioxidant available from Ciba Specialty Chemicals, Tarrytown, N.Y.

Open cell polyurethane foam, available from 3M, St. Paul, Minn.

Anti-Microbial Performance Tests

2 Hours % Live Bacteria Test

The effectiveness of a sample was tested using a L-7012, Bacterial Viability Kit, available from Molecular Probes (Eugene, Oreg.). The procedure is outlined below using the red, propidium iodide dye, and green, SYTO 9 dye, contained in the kit to stain the live and dead bacteria.

Preparation of bacteria solution: Staphylococcus aureus bacteria and E. coli were grown in Trypticase (Tryptic) Soy Broth (TSB) medium overnight. Bacteria were concentrated by centrifugation at 10,000× gravity for 15 minutes (min). Supernatant was removed and the pellet was re-suspended in MilliQ water (filtered through a 0.2 μm pore-size filter) or in Butterfield phosphate buffer (from Hardy Diagnostics, Santa Maria, Calif.). Bacteria solution was diluted to the desired bacteria concentration (10⁷ cells/milliliters) by measuring the optical density (OD) at 670 nm. For a control experiment, the bacteria solution was incubated with 70% isopropyl alcohol at room temperature for 1 hour (hr) to measure the killed bacteria control. Different volume of live and dead bacteria solutions were mixed to generate a range of percent live solution for calibration purposes.

Sample preparation: All prototypes were prepared by punching out a 0.125 inch (0.05 cm) to 1-inch (2.54-cm) diameter samples using a stainless steel punch; sometimes as indicated in the examples a 1-inch (2.54 cm) disk was further cut with scissors in eighths and then evaluated. The amount of sample was weighed, and then transferred to 50 milliliters (mL) sterile conical tubes.

Bacteria labeling and Anti-microbial testing: 7 mL of bacteria solution at initial concentration of approximately 1×10⁸ bacteria/mL were pipetted into a 50 mL conical tube containing the sample. At the specified time (e.g., 2 hr), 50 micro-liter (μL) of the supernatant was pipetted into fluorescent measurement tube which already contained 450 μL of MiliQ water and premixed green dye and red dye solution (1.5 μL dye mixture for 500 μL bacteria solution) was added and the mixture was incubated for 15 minutes in the dark at room temperature. These solutions were then measured by flow cytometry. Cell viability was measured using the BD FACSCaliber flow cytometer (made by Becton Dickinson & Company, Franklin Lakes, N.J.). The flow cytometer is equipped with an argon-ion laser at 488 nanometers (nm) and 15 milliwatts (mW) output. Data acquisition and analysis were controlled using CellQuest software and PBPAC hardware interface. The light path contained a 488/10 nm blocking filter, then a 530/30 nm filter before the green PMT and a 585/42 nm long pass filter before the red PMT. The sampling rate was around 3000-7000 particles/second. The sheath fluid was FACSFlow by Becton Dickinson. The instrument voltage was 5.5 Volt.

The live cell and dead bacteria responses were established with the 100% live cell and 100% dead cell (for killed bacteria, bacteria solution was incubated with 70% isopropyl alcohol at room temperature for 1 hr) samples. Different volumes of live and dead bacteria solutions were mixed to generate a range of percent live solutions for calibration purposes. The sample results for bacteria killing ability were interpolated from the standard curve generated from calibration samples. Total bacteria concentration was determined by the measuring of the OD at 670 nm of the bacteria solution.

Zone of Inhibition Test

Anti-microbial performance was measured using a Zone of Inhibition test (ZOI) that was performed by the following method. Mueller-Hinton agar was prepared, sterilized and tempered in a water bath at 48-50° C. A suspension of bacteria in sterile phosphate-buffered water was prepared with approximately 10⁸ CFU/ml. The agar was inoculated with a bacterial suspension of bacteria to an approximate concentration of 10⁵ CFU/ml (1:1000). The inoculated agar was swirled to mix and pipetted (˜14 ml) into sterile Petri dishes (15×100 mm). The seeded agar was allowed to set for about 20 minutes to harden. An alcohol-disinfected die and cutting board were used to cut textile samples to desired size. Sterile forceps were used to place the samples onto the seeded, hardened agar in center of plate. The plate was then placed into an incubator at 35-37° C. for overnight (16-24 hours) incubation. After incubation the clear zones, where no visible colonies formed, were measured in mm with calipers.

The zone of inhibition (ZOI) is then calculated by the following equation
ZOI=[diameter of clear zone (mm)−diameter of sample (mm)]/2
Saline Absorbency Test

Samples were soaked in 0.85% by weight sodium chloride solution (saline). The samples were removed from the saline at various times and were lightly dabbed with a paper towel. The weight was recorded. The weight of saline absorbed per weight of dry coating was calculated using the following equation: (weight saline absorbed)=[(saline swollen weight)−(dry sample weight)]/(dry sample weight).

### Example 1

A clear solution of 1% silver (II) oxide and 5% ammonium carbonate in water was prepared by stirring the mixture until the silver (II) oxide was fully dissolved. A 7.62×5.08 cm nonwoven cotton gauze was dipped in the solution for five seconds, removed and patted with a paper towel to remove excess solution. The coated gauze was then dried in a 150° C. oven for ten minutes. After drying, the gauze turned a deep brown color.

When dipped in saline, the cotton gauze coated with silver oxide absorbed 4.89 grams saline per gram dressing. As a comparison, a cotton gauze sample without silver oxide coating absorbed 4.75 grams saline per gram dressing.

Zone of Inhibition tests were run on three 7 mm samples of the silver oxide-coated cotton gauze over 9 days. At the end of each 24-hour period, the samples were evaluated, removed from the agar plate and transferred to a freshly inoculated agar plate.

Zone of Inhibition results are shown in Table 1 below:

TABLE 1

| | | Growth under the |
|---|---|---|
| Day | ZOI (mm) | sample disc |
| 1 | 3 | None |
| 2 | 2 | None |
| 3 | 2 | None |
| 4 | 1.5 | None |
| 5 | 1.5 | None |
| 6 | 1.5 | None |
| 7 | .5 | None |
| 8 | 0 | Slight |
| 9 | 0 | Moderate |

### Example 2

A solution of 30 parts of silver (I) oxide, 100 parts ammonium carbonate, and 2870 parts water were mixed in a glass jar until the silver (I) oxide was completely dissolved. The solution was gravure coated at 100 g/m² at 1.6 m/min on a nonwoven cotton. The coated nonwoven cotton was heated in an oven at 160° C. for 5 minutes. The dry coating was light brown.

### Example 3

The solution was prepared as in Example 2 except that the solution was coated on woven cotton. After microwave digestion of the woven cotton gauze, analysis by an ion chromatograph (model, source) showed no detectable ammonium ion.

Zone of Inhibition tests were run on three layers of 10 mm sample. The ZOI after 24 hours was 3.75 for S. aureus and 2.85 for E. coli.

### Example 4

Same as Example 2 except that the solution was coated on a polyester knit. The dried coating was light grey.

### Example 5

Same as Example 2 except that silver (II) oxide was used.

### Example 6

Nonwoven cotton gauze was dipped in a solution comprising 1% Ag₂O and 5% ammonium pentaborate in water. Excess solution was squeezed from the dipped gauze, and the gauze was weighed. The total solution weight absorbed by the gauze sample was 2.5 grams. When divided by the area of the gauze, the total solution uptake was 0.024 grams/cm². The silver compound concentration on the gauze was 0.24 mg/cm².

The gauze was dried in 150° C. oven for 10 minutes. After drying, the gauze turned dark brown in color. The ZOI after 24 hours was 1.5 mm.

### Example 7

Nonwoven cotton gauze was dipped in a solution comprising 2% silver carbonate, 5% ammonium acetate and 1.5% ammonia with the balance water. Excess solution was squeezed from the dipped gauze, and the gauze was weighed. The total solution weight absorbed by the gauze sample was 2.24 grams. When divided by the area of the gauze, the total solution uptake was 0.028 grams/cm². The total silver compound concentration on the gauze was 0.56 mg/cm².

The gauze was dried in 150° C. oven for 10 minutes. After drying, the gauze turned medium brown in color. The ZOI after 24 hours was 2 mm.

### Example 8

Polyurethane foam was dipped in a solution comprising 1% silver (II) oxide (AgO) and 5% ammonium carbonate in water. Excess solution was squeezed from the dipped foam, and the foam was weighed. The total solution weight absorbed by the foam sample was 6 grams. When divided by the area of the sample, the total solution uptake was 0.095 grams/cm². The total silver compound concentration on the gauze was 0.95 mg/cm².

The foam was dried in 120° C. oven for 10 minutes. After drying, the foam turned brown in color. The ZOI after 24 hours was 2 mm.

### Example 9

Polyurethane foam was dipped in a solution comprising 1% silver sulfate and 5% ammonium carbonate in water. Excess solution was squeezed from the dipped foam, and the foam was weighed. The total solution weight absorbed by the foam sample was 3.2 grams. When divided by the area of the sample, the total solution uptake was 0.051 grams/cm². The total silver compound concentration on the foam was 0.51 mg/cm².

The foam was dried in 120° C. oven for 10 minutes. After drying, the foam turned brown in color. The ZOI after 24 hours was 1.5 mm.

### Example 10

Woven cotton gauze was ink jet coated with a solution comprising 4% THV 200 fluorothermoplastic (available from Dyneon, LLC, Oakdale, Minn.) in Methylethyl ketone solution (available from Sigma Aldrich, Milwaukee, Wis.) using the Xaar XJ128-200 piezo printhead (Available form Xaar Ltd., Cambridge, England) at 300×300 dpi.

Nonadherency of the coated gauze was evaluated using a 2 inch piece of Scotch™ Magic Tape (available from 3M, St. Paul, Minn.) by applying the tape to the coated gauze, rolling once, and removing by hand. The tape removed easily without pulling fibers. Gauze without the THV coating resisted pull, and fibers were pulled off when the tape was removed.

The gauze coated with silver solution of Example 1 was placed between the THV coated gauze and sealed at the edges using double-stick tape. The silver-nonadherent gauze construction absorbed 3.28 grams of saline. Using 7 mm samples of the gauze construction, the ZOI after 24 hours was 2.5 mm.

### Example 11

The coated gauze of Example 1 was placed between two sheets of woven 100% nylon fabric (available from JoAnn Fabrics, Woodbury, Minn.) and sealed at the edges using double-stick tape. The silver-nylon gauze construction absorbed 3.46 grams of saline. Using 7 mm samples of the construction, the ZOI after 24 hours was 1.5 mm.

### Example 12

A hydrocolloid dressing, under the trade name Tegasorb™ (available from 3M, St. Paul, Minn.) was dipped in a clear silver solution prepared with 100 parts of silver (I) oxide, 337 parts of ammonium carbonate, and 3000 parts of de-ionized water. The dressing was soaked in the silver solution for two minutes, contacting only the hydrocolloid material. The coated hydrocolloid substrate was placed in an oven at 100° C. for 30 minutes.

The coated dressing was tested using the % Live Bacteria Test. Samples having a diameter of 12.7 mm were placed in contact with 7 mls of bacterial solution having approximately 10⁸ counts of S. aureaus. At 30 minutes the % Live results were 60.5, and at 2 hours the % Live results were 0.72.

### Example 13

A nonadherent hydrocolloid dressing was prepared based on a Styrene-isoprene-styrene gel and Salcare™ SC91 hydrocolloid. KRATON D1124K styrene-isoprene-styrene (SIS) pellets were gravimetrically fed into the feed throat (barrel section 1) of a Werner Pfleiderer ZSK30 co-rotating twin-screw extruder (TSE) having a 30 mm diameter and 15 barrel sections.

Each temperature zone was a combination of two barrel sections (e.g., Zone 1 corresponded to barrel sections 2 and 3). Barrel section 1 was controlled at full cooling capacity for all SIS gel lots. A powdered antioxidant (IRGANOX 1010) was also gravimetrically fed into barrel section 1. KAYDOL mineral oil was heated and added to the TSE as described in International Publication No. WO 97/00163. The disclosed compounding process provides a method for making a gel by melting of the SIS elastomer followed by addition of the heated mineral oil. Heated mineral oil was sequentially injected into barrel sections 4, 6, 8, 10 and 12, respectively. The TSE screw speed was controlled to 400 revolutions per minute (rpm). The TSE temperature profile was controlled to 204° C., 227° C., 227° C., 204° C., 182° C., 171° C., and 93° C. for zones 1-7, respectively. The heated oil injections were controlled to 204° C., 204° C., 204° C., 177° C., and 177° C., respectively. Table 2 contains the material flow rates and Table 3 contains the compositional information for the SIS gel.

TABLE 2
SIS gel flow rates

| | Barrel Section(S) and Oil | | | | | | | |
|---|---|---|---|---|---|---|---|---|
| | addition number | | | | | Total | | |
| | and Rate (g/min) | | | | | KAYDOL | IRGANOX | Total |
| SIS | S4 | S6 | S8 | S10 | S12 | Oil | 1010 | Flow Rate |
| (g/min) | Oil 1 | Oil 2 | Oil 3 | Oil 4 | Oil 5 | (g/min) | (g/min) | (g/min) |
| 227 | 74 | 100 | 120 | 120 | 108 | 522 | 8 | 757 |

TABLE 3
SIS gel composition

| | | KAYDOL | IRGANOX |
|---|---|---|---|
| SIS | SIS | oil | 1010 |
| Type | (wt- %) | (wt- %) | (wt- %) |
| Radial | 30.0 | 69.0 | 1.0 |

The pre-compounded SIS gel was combined with SALCARE SC91 in a Haake 25 mm diameter, fully intermeshing counter-rotating TSE. The SIS gel was re-melted in a Bonnot extruder operating at 127° C., and injected at 22.8 grams per minute into barrel section 2 of the TSE. SALCARE SC91 inverse emulsion was injected at ambient temperature into barrel section 4 at 15.2 grams per minute (g/min) using a Zenith gear pump. The TSE was controlled at 300 rpm screw speed and 121° C. temperature. The total material throughput was 38.0 grams per minute. The SIS gel/SALCARE SC91 blend was discharged out of the TSE into a transport hose using a Zenith gear pump. A transport hose conveyed the molten gel blend to a 0.15 meter (m) wide single orifice film die. The transport hose and die were both controlled to 121° C. The molten gel blend was extruded into a nip formed by two gapped and polished steel rolls controlled to 110° C. A polyester (PET) knitted fabric having 0.8 mm by 0.7 mm (0.56 mm²) rectangular open apertures, 0.20 millimeter (mm) thickness and 0.15 meter (m) width was also fed into the nip at 1.4 m/min speed. As the fabric exited the nip, the gel-coated article was cooled in air before being wound up with an inserted paper release liner. After air-cooling to ambient temperature a coated fabric having 0.75 mm by 0.6 mm (0.45 mm²) rectangular open apertures was obtained. Table 4 contains the process conditions and Table 5 contains the compositional information for the dressing:

TABLE 4
Process conditions

| SIS Gel Input | SALCARE Input | Steel Roll | Coating | Coating |
|---|---|---|---|---|
| (barrel section | (barrel section | Gap | Speed | Weight |
| number) | number) | (mm) | (m/min) | (g/m²) |
| 2 | 4 | 0.25 | 2.1 | 78 |

TABLE 5
Composition

| | IRGANOX | SALCARE | |
|---|---|---|---|
| SIS | 1010 | SC91 | KAYDOL oil |
| (wt- %) | (wt- %) | (wt- %) | (wt- %) |
| 18.0 | 0.6 | 40.0 | 41.4 |

The nonadherent dressing was dipped in a clear silver solution prepared with 100 parts of silver (I) oxide, 337 parts of ammonium carbonate, and 3000 parts of de-ionized water. The dressing was soaked in the silver solution for two minutes, contacting only the hydrocolloid material. The coated hydrocolloid dressing was placed in an oven at 100° C. for 30 minutes.

The coated dressing was tested using the % Live Bacteria Test. Samples having a diameter of 12.7 mm were placed in contact with 7 mls of bacterial solution having approximately 10⁸ counts of S. aureaus. At 30 minutes the % Live results were 0.92, and at 2 hours the % Live results were 0.04.

### Example 14

A solution of 1.3% silver (I) oxide, 4.4% ammonium carbonate, and 94.3% water were mixed in a glass jar until the silver (I) oxide was completely dissolved. The solution was gravure coated at 100 g/m² at 1.6 m/min on a nonwoven cotton. The coated nonwoven cotton was heated in an oven at 160° C. for 5 minutes.

The coated dressing was tested using the % Live Bacteria Test. Samples having a diameter of 12.7 mm were placed in contact with 7 mls of bacterial solution having approximately 10⁸ counts of S. aureaus. At 30 minutes the % Live results were 2.91, and at 2 hours the % Live results were 0.07.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A method of coating silver compounds on a substrate, the method comprising:
combining a sparingly soluble silver-containing compound with an ammonium-containing compound to form an aqueous solution,
coating the solution on a substrate,
and drying the coated substrate.

2. The method of claim 1, wherein the solution has a pH of about 9.

3. The method of claim 1 wherein the solution is formed at less than 40° C.

4. The method of claim 1, wherein the solution is coated at less than 40° C.

5. The method of claim 1, wherein the silver-containing compound is selected from the group consisting of silver chloride, silver sulfate, silver carbonate, silver oxide, silver stearate, silver phosphate, silver thiocyanate.

6. The method of claim 5 wherein the silver-containing compound is silver oxide.

7. The method of claim 1, wherein the ammonium-containing compound is selected from the group consisting of ammonium carbonate, ammonium pentaborate and ammonium acetate.

8. The method of claim 7 wherein the ammonium-containing compound is ammonium carbonate.

9. The method of claim 1, wherein the silver-containing compound forms a silver-ammonium complex when combined with the ammonium-containing compound.

10. The method of claim 1, wherein the silver-containing compound remains on the substrate after drying the substrate while the remainder of the coating is volatilized.

11. The method of claim 1, wherein the ammonium-containing compound is essentially all removed after drying the substrate.

12. The method of claim 1, further comprising the step of adding an oxidizing agent to the solution.

13. The method of claim 1, further comprising the step of adding an oxidizing agent to the coated substrate.

14. The method of claim 1, wherein the substrate is selected from the group consisting of a nonwoven gauze, a woven gauze, a polyester fiber, a foam, a film and a hydrocolloid.

15. A method of coating silver compounds on a substrate, the method comprising:
combining silver oxide with ammonium carbonate to form an aqueous solution,
coating the solution on a substrate,
and drying the coated substrate.

16. The method of claim 15, wherein the solution has a pH of about 9.

17. The method of claim 15, wherein the solution is formed at less than 40° C.

18. The method of claim 15, wherein the solution is coated at less than 40° C.

19. The method of claim 15, wherein the silver oxide forms a silver-ammonium complex when combined with the ammonium carbonate.

20. The method of claim 15, wherein the silver oxide is the only compound from the solution that remains on the substrate after drying the substrate.

21. The method of claim 15, wherein the ammonium carbonate is removed after drying the substrate.

22. The method of claim 15, further comprising the step of adding an oxidizing agent to the solution.

23. The method of claim 15, further comprising the step of adding an oxidizing agent to the coated substrate.

24. The method of claim 15, wherein the substrate is selected from the group consisting of a nonwoven gauze, a woven gauze, a polyester fiber, a foam, a film and a hydrocolloid.

25. An article made by the method of claim 1 wherein the article impregnated with sparingly soluble silver-containing compound is essentially free of the ammonium compound or residual components of the ammonium compound and the silver-containing compound introduced during the application of the solution.

26. An article made by the method of claim 15 wherein the article impregnated with silver oxide is essentially free of compounds introduced during the application of the solution other than the silver oxide.

27. A method of coating silver compounds on a substrate, the method comprising:
combining silver oxide with an ammonium-containing compound to form an aqueous solution,
adding an oxidizing agent in an effective amount to increase the valence state of the silver oxide,
coating the solution on a substrate,
and drying the coated substrate.

28. The method of claim 27, wherein the solution has a pH of about 9.

29. The method of claim 27, wherein the solution is formed at less than 40° C.

30. The method of claim 27, wherein the solution is coated at less than 40° C.

31. The method of claim 27, wherein the ammonium-containing compound is selected from the group consisting of ammonium carbonate, ammonium pentaborate and ammonium acetate.

32. The method of claim 31 wherein the ammonium-containing compound is ammonium carbonate.

33. The method of claim 27, wherein the silver oxide forms a silver-ammonium complex when combined with the ammonium-containing compound.

34. The method of claim 27, wherein the silver oxide is the only compound from the solution that remains on the substrate after drying the substrate.

35. The method of claim 27, wherein the substrate is selected from the group consisting of a nonwoven gauze, a woven gauze, a polyester fiber, a foam, a film and a hydrocolloid.

36. The method of claim 1, wherein the composition is stable.

37. A wound dressing made by the method of claim 1.

38. A wound dressing made by the method of claim 15.

39. A wound dressing made by the method of claim 27.

40. A medical article comprising a porous substrate impregnated with one or more sparingly soluble silver compounds, wherein the medical article has less than 1 N/cm peel strength to steel and does not adhere to wound tissue.

41. The medical article of claim 40, wherein the medical article is capable of absorbing saline at least 100% of its dry weight.

42. The medical article of claim 40, wherein the medical article is capable of absorbing saline at least 200% of its dry weight.

43. The medical article of claim 40, wherein the porous substrate is nonadherent.

44. The medical article of claim 40, wherein the porous substrate is covered on one or more sides by a nonadherent layer.
